(19) United States
(12) Patent Application Publication
(10) Pub. No.: US 2025/0262369 A1
(43) Pub. Date: Aug. 21, 2025

(54) SINGLE OR DOUBLE LUMEN SYSTEM

(71) Applicant: Stavro Medical, Inc., Martinez, CA (US)

(72) Inventors: Lakhmir Singh Chawla, Martinez, CA (US); Edward Joseph Plute, III, Denver, CO (US)

(21) Appl. No.: 19/195,993

(22) Filed: May 1, 2025

Related U.S. Application Data
(63) Continuation of application No. PCT/US2023/078338, filed on Nov. 1, 2023.
(60) Provisional application No. 63/423,144, filed on Nov. 7, 2022.

(51) Int. Cl. A61M 1/36; A61M 60/109; A61M 60/232; A61M 60/40; A61M 60/546

(52) U.S. Cl. CPC A61M 1/3672; A61M 60/109; A61M 60/232; A61M 60/40; A61M 60/546

(57) ABSTRACT

The disclosure provides methods and systems to improve the extracorporeal processing and treatment of blood.

### CROSS-REFERENCE TO RELATED APPLICATIONS

This application claims priority to PCT Application No. PCT/US2023/078338, filed Nov. 1, 2023, which application claims priority to U.S. Patent Application No. 63/423,144, filed Nov. 7, 2022, the contents both of which are hereby incorporated by reference in its entireties for all purposes.

### BACKGROUND

In extracorporeal blood treatments, blood from a patient (e.g., human or animal) is withdrawn for treatment processing, and the processed blood is subsequently returned to the patient. Conventional extracorporeal blood treatment methods include, but are not limited to, apheresis, plasmapheresis, hemoperfusion (HPF), and renal replacement therapies (RRT), such as ultrafiltration (UF), hemodialysis (HD), hemofiltration (HF), and hemodiafiltration (HDF). Blood-based RRT systems generally require access to the patient's vascular stream. In conventional RRT systems, sufficient clearance of waste molecules and/or fluids from the processed blood requires a certain blood flow rate through the treatment module.

Embodiments of the disclosed subject matter improve the extracorporeal processing and treatment of blood and offers other advantages as well.

### BRIEF SUMMARY

In one embodiment, the present disclosure provides a blood treatment method comprising:
(a) conveying a volume of blood via a first conduit from a first vascular access of a patient to a blood chamber at a first flow rate, the first conduit having a first lumen;
(b) conveying the blood from the blood chamber through an extracorporeal therapeutic device at a second flow rate to perform an extracorporeal treatment on the blood and returning the treated blood to the blood chamber; and
(c) returning the blood from the blood chamber to a second vascular access of the patient at a third flow rate via a second conduit, the second conduit having a second lumen;
wherein the second flow rate is decoupled from both the first and third flow rates.

In another embodiment, the present disclosure provides a blood treatment method comprising:
(a) conveying a volume of blood via a first conduit from a first vascular access of a patient to a first T-junction at a first flow rate, the first conduit having a first lumen;
(b) conveying the blood from the first T-junction through an extracorporeal therapeutic device at a second flow rate to perform an extracorporeal treatment on the blood and returning the treated blood to a second T-junction; and
(c) returning the blood from the second T-junction to a second vascular access of the patient at a third flow rate via a second conduit, the second conduit having a second lumen, or alternatively,
(d) conveying the blood from the second T-junction to a blood chamber to reprocess the blood in the extracorporeal treatment, wherein the second flow rate is decoupled from both the first and third flow rates.

In yet another embodiment, the present disclosure provides a blood treatment method comprising:
(a) conveying a volume of blood via a conduit from a vascular access of a patient to a T-junction at a first flow rate, the conduit having a lumen;
(b) conveying the blood from the T-junction through an extracorporeal therapeutic device at a second flow rate to perform an extracorporeal treatment on the blood and returning the treated blood to a blood chamber;
(c) returning the blood from the blood chamber to the vascular access of the patient at a third flow rate, wherein the second flow rate is decoupled from both the first and third flow rates.

Additional objects, advantages and embodiments of the disclosed subject matter will become apparent from the following description when considered in conjunction with the accompanying drawings.

### BRIEF DESCRIPTION OF THE DRAWINGS

FIG. 1 is a simplified schematic diagram of a generalized blood treatment system according to one or more embodiments of the disclosed subject matter.

FIG. 2A is a simplified schematic diagram of a generalized blood treatment system according to one or more embodiments of the disclosed subject matter.

FIG. 2B is a simplified schematic diagram of a generalized blood treatment system according to one or more embodiments of the disclosed subject matter.

FIG. 3 is a simplified schematic diagram of a generalized blood treatment system according to one or more embodiments of the disclosed subject matter.

### DETAILED DESCRIPTION

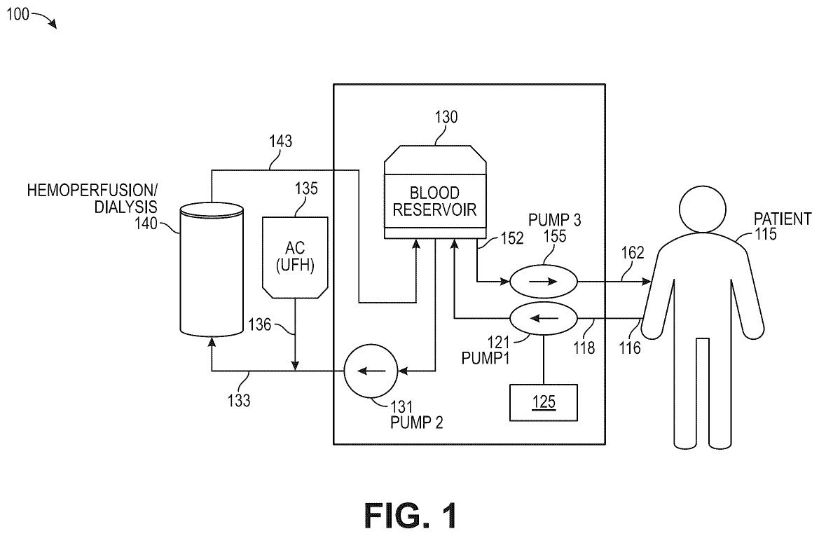

FIG. 1 illustrates an embodiment of the present disclosure. System 100 includes a double lumen configuration with a 4-port blood chamber, reservoir, or bag 130. The system 100 transfers blood to/from patient 115 and processes the blood for treatment. For example, a vascular access 116 is coupled to a single-lumen conduit 118 to provide blood from patient or subject 115 to the blood chamber 130 using a first blood pump 121. The blood pump can be for example, a centrifugal pump, a Harvard apparatus, or a syringe pump. The vascular access 116 can comprise a needle, catheter, or any other device for connecting to the patient's vascular system known in the art. The extracorporeal therapeutic device or treatment module (used interchangeably) 140 is designed or configured to affect an extracorporeal treatment on blood passing therethrough, for example, filtration, hemoperfusion, plasma separation, dialysis treatment including, but not limited to, ultrafiltration, hemofiltration (HF), hemodiafiltration (HDF), hemodialysis (HD), or hemoperfusion (HPF).

In certain aspects, blood leaving patient 115 fills the blood chamber 130 using conduit 118 and first blood pump 121. The unprocessed blood leaves blood chamber 130 via conduit 133 using second blood pump 131. Treatment module 140 treats the blood and is returned to blood chamber using conduit 143. A fluid/drug module 135 with an associated supply conduit 136 can be used to infuse for example, anticoagulant into conduit 133. For example, when the patient has not otherwise been dosed with an anticoagulant, controller 125 can instruct the addition of an appropriate anticoagulant, such as, but not limited to, heparin, citrate-based anticoagulants, nafamostat, or epoprostenol via conduit 136 from fluid/drug module 135.

In certain instances, controller 125 controls fluid/drug module 135, first blood pump 121, a second blood pump 131, a third blood pump 155, blood chamber 130, treatment module 140 and fluid/drug module 135 and various valves or other fluid control components (not shown) to pump secondary fluid and/or anticoagulant from fluid/drug module 135 via input conduits 136 to conduit 133, and into the patient's blood.

In operation, blood is withdrawn from patient 115 via access 116 and conveyed to chamber 130 for temporary storage until treatment processing. For example, controller 125 can control first blood pump 125 and various valves or other fluid control components (not shown) to pump the blood from patient 115 along single-lumen conduit 118 to blood chamber 130 at a first flow rate. The blood conveying is continued until a predetermined blood volume is obtained in the chamber 130. The predetermined blood volume may be adjustable based on a size of patient 115, for example, 2-7% or 1-15% such as 1, 2, 3, 4, 5, 6, 7, 8, 9, 10, 11, 12, 13, 14 or 15% of a total blood volume of patient 115. For example, the predetermined blood volume may be 10-300 ml, or 10 ml to 1000 ml and may be set by the patient 115 or system operator. The treatment can be repeated until an entire body blood volume is treated (e.g., about 5 liters).

The controller 125 can monitor the volume of blood in chamber 130 and determine whether the predetermined blood volume has been met. For example, a weight of chamber 130 and contents therein can be monitored by a highly accurate weight sensor e.g., a gravity scale. Because the blood volume in chamber 130 is relatively small (e.g., less than 300 ml), the chamber 130 can be weighed very accurately to avoid incorrect volume correlations. For example, the weight sensor may have an accuracy down to ±1 gram or less. Those of skill in the art will know of other sensors to measure fluid level including, but not limited to, floats, gauges, capacitive level sensors, light sensors and other volume or weight sensors, which can be used.

Controller 125 can then correlate changes in weight of chamber 130 to changes in fluid/blood volume therein. Controller 125 can also correlate changes or the presence of a signal when other volume levels sensors are used. In some aspects, weight sensor provides signals to controller 125 in real-time during fill of chamber 130. The sensor and/or controller 125 may thus be configured to compensate for any weight fluctuations due to fluid dynamics/vibration within the chamber during the blood flow.

The present disclosure provides a blood treatment method comprising:
(a) conveying a volume of blood via a first conduit from a first vascular access of a patient to a blood chamber at a first flow rate, the first conduit having a first lumen;
(b) conveying the blood from the blood chamber through a treatment module at a second flow rate to perform an extracorporeal treatment on the blood and returning the treated blood to the blood chamber; and
(c) returning the blood from the blood chamber to a second vascular access of the patient at a third flow rate via a second conduit, the second conduit having a second lumen;
   wherein the second flow rate is decoupled from both the first and third flow rates.

The first flow rate is dependent on the rate of the first blood pump 121, which fills the blood chamber 130 by conveying blood from the patient 115 via conduit 118. The second flow rate is determined by the second blood pump 131, which removes blood from the blood chamber 130 via conduit 133 and through the extracorporeal therapeutic device or treatment module 140. The second flow rate conveys blood from treatment module 140 through conduit 143 back to blood chamber 130. A third flow rate is determined by the third blood pump 155, which removes blood from the blood chamber 130 via conduit 152 back to the patient via conduit 152, through lumen 162.

In certain aspects, the second flow rate is faster than the combination of both the first and the third flow rates. Such flow rates allow the blood to be treated before being returned to the blood chamber. Since the treatment processing is decoupled from the blood withdrawal and infusion, a lower blood flow rate can be used for withdrawal/infusion of blood, thereby enabling a smaller bore/diameter for the needle or lumen providing access to the patient's vascular system. Although the present disclosure uses “blood” as an exemplary body fluid, those of skill in the art will recognize that the systems and methods of the present disclosure are also useful for other body fluids such as blood, lymph, ascites, abdominal fluid, pleural fluid, organ fluid, spinal fluid, intestinal fluid or water. Similarly, although “vascular access” is an exemplary embodiment, a skilled artisan will recognize that abdominal access is needed for ascites, spinal canal access is needed for spinal fluid, and lymphatic access is need for lymph. In some aspects, processed blood can be returned to the blood chamber and repeatedly processed by the treatment module (e.g., passing through the treatment module multiple times) to further improve for example, solute clearance, before returning to patient 115.

In some aspects, the systems and methods herein utilizes a small lumen 116 (e.g., smaller than either 7 French, such as 6, 5, 4, or 3 French or 17 gauge such as 16, 15, 14, 13, 12, 11, or 10 gauge) to draw, at a first flow rate, a “micro” batch of blood or body fluid (e.g., about 10-300 ml, or 2-7% such as about 2, 3, 4, 5, 6, or 7% of the patient's 115 total blood volume) into a chamber 130. The volume of body fluid can be, for example, about 10 ml-100 ml, 10 ml to 200 ml, 10 ml to 300 ml, 10 ml to 400 ml, 10 ml to 500 ml, 10 ml to 600 ml, 10 ml to 700 ml, 10 ml to 800 ml, 10 ml to 900 ml or 10 ml-1000 ml. Once in the chamber 130, the batch of blood can be circulated at a higher second flow rate through a treatment module 140 (the treatment circuit), such as hemofilter, hemodialyzer, or hemoperfusion device, thereby enabling efficient small and middle molecule clearance. After sufficient circulations, the blood is returned 152, at a third flow rate (which may be the same as or different from the first flow rate) to the patient via a lumen 162. The cycle (within the treatment circuit) can then be repeated multiple times, for example, to process an entire blood volume of the patient, about 5 liters.

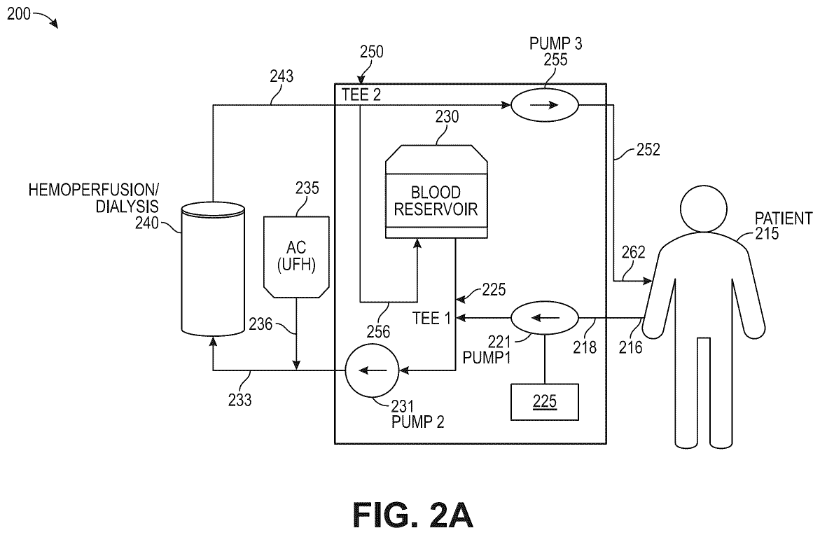

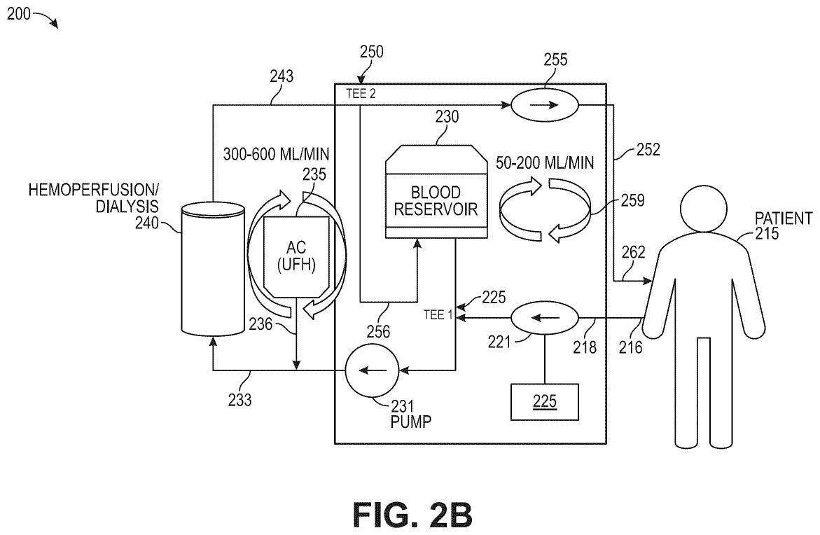

Turning now to FIG. 2A, disclosed therein is another embodiment of the present disclosure. The disclosure provides a blood treatment method comprising:
(a) conveying a volume of blood via a first conduit from a first vascular access of a patient to a first T-junction at a first flow rate, the first conduit having a first lumen;
(b) conveying the blood from the first T-junction through an extracorporeal therapeutic device at a second flow rate to perform an extracorporeal treatment on the blood and returning the treated blood to a second T-junction; and
(c) returning the blood from the second T-junction to a second vascular access of the patient at a third flow rate via a second conduit, the second conduit having a second lumen, or alternatively,
(d) conveying the blood from the second T-junction to a blood chamber to reprocess the blood in the extracorporeal treatment, wherein the second flow rate is decoupled from both the first and third flow rates.

System 200 includes a double lumen configuration with a 2-port blood chamber, reservoir, or bag 230. The system 200 transfers blood to/from patient 215 and processes the blood for treatment. For example, a vascular access 216 is coupled to a single-lumen conduit 218 to provide blood from patient or subject 215 to a first T-junction 225 at a first flow rate. In certain aspects, the blood leaving patient 215 is conveyed to the first T-junction using conduit 218 and a first blood pump 221. The vascular access 216 can comprise a needle, catheter, or any other device for connecting to the patient vascular system known in the art. The treatment module 240 is designed to affect an extracorporeal treatment of blood passing thereto, for example, a dialysis treatment including, but not limited to, hemofiltration (HF), hemodiafiltration (HDF), hemodialysis (HD), or hemoperfusion (HPF).

In certain aspects, blood or other body fluid is conveyed from the first T-junction 225 through an extracorporeal therapeutic device or treatment module 240 using conduit 233 at a second flow rate determined by a second blood pump 231 to perform an extracorporeal treatment on the blood and returning the treated blood to a second T-junction 250. Blood or other body fluid is moved or conveyed from the treatment module 240 via conduit 243 to the second T-junction 250. Treatment module 240 treats the blood and is returned to blood chamber using conduit 243. A fluid/drug module 235 with an associated supply conduit 236 can be used to infuse for example, anticoagulant into conduit 233. For example, when the patient has not otherwise been dosed with an anticoagulant, controller 225 can instruct the addition of an appropriate anticoagulant, such as, but not limited to heparin, citrate-based anticoagulants, nafamostat, or epoprostenol via conduit 236 from fluid/drug module 235.

Treatment module 240 treats the blood and is conveyed to blood chamber 230 using conduit 243.

In certain aspects, the method provides returning the blood from the second T-junction 250 to a second vascular access of the patient 262 at a third flow rate via a second conduit 252, the second conduit having a second lumen 262. The third flow rate is determined by a third blood pump 255.

Alternatively, or in addition, the method provides conveying the blood from the second T-junction 250 to a blood chamber 230 to reprocess or repeat the blood in the extracorporeal treatment module 240. In certain aspects, the second flow rate is decoupled from both the first and third flow rates.

Once in the chamber 230, the batch of blood can be circulated at a higher second flow rate through a treatment module 240 (the treatment circuit). After sufficient circulations, the blood is returned 252, at a third flow rate (which may be the same as or different from the first flow rate) to the patient via a lumen 262. The cycle can then be repeated multiple times, for example, to process an entire blood volume of the patient.

FIG. 2B shows system 200 with rates associated with the first, second and third flow rates. For example, the treatment circuit comprises the first T-junction 255, the second blood pump 231, conduit 233, treatment module 240, conduit 243, second T-junction 250, conduit 256 and blood chamber 230. The treatment circuit, which comprises the second flow rate, is decoupled from both the first and third flow rates.

The second flow rate, which is exemplified by the arrows surrounding 235, is between about 300 ml/min to about 600 ml/min. The rates include, about 300 ml/min, 325 ml/min, 350 ml/min, 375 ml/min, 400 ml/min, 425 ml/min, 450 ml/min, 475 ml/min, 500 ml/min, 525 ml/min, 550 ml/min, 575 ml/min, and 600 ml/min.

The first and third flow rates 259 are between about 50 ml/min to about 500 ml/min, such as about 50 ml/min, 75 ml/min, 100 ml/min, 125 ml/min, 150 ml/min, 175 ml/min, and about 200 ml/min. The first and third flow rates can be the same or different.

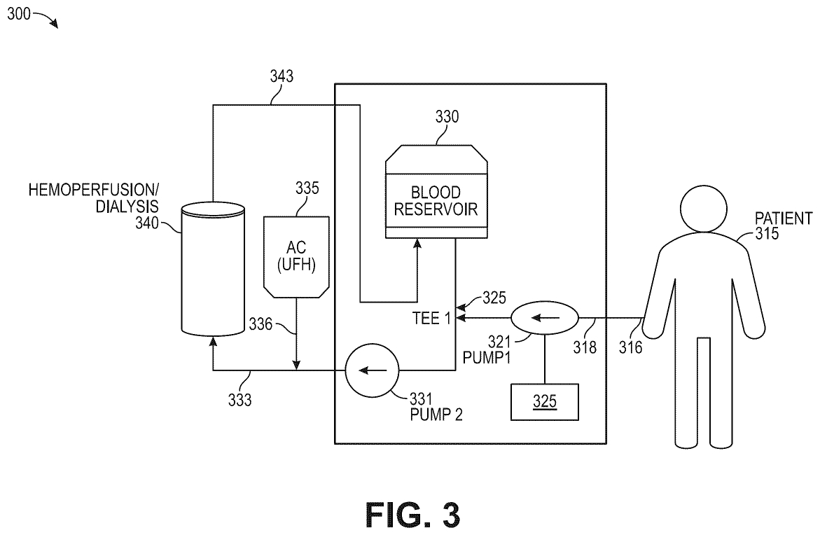

FIG. 3 illustrates yet another embodiment of the present disclosure. System 300 includes a single lumen configuration with a 2-port blood chamber, reservoir 330 or bag. The disclosure provides a blood treatment method comprising:
(a) conveying a volume of blood via a conduit from a vascular access of a patient to a T-junction at a first flow rate, the conduit having a lumen;
(b) conveying the blood from the T-junction through an extracorporeal therapeutic device at a second flow rate to perform an extracorporeal treatment on the blood and returning the treated blood to a blood chamber;
(c) returning the blood from the blood chamber to the vascular access of the patient at a third flow rate, wherein the second flow rate is decoupled from both the first and third flow rates.

System 300 transfers blood to/from patient 315 and processes the blood for treatment. For example, a vascular access 316 is coupled to a single-lumen conduit 318 to provide blood from patient or subject 315 to a T-junction 325 at a first flow rate, the conduit having a lumen. The first blood pump 321 conveys blood from the patient 315 to the T-junction 325 within conduit 318. Blood is conveyed from the T-junction 325 through a treatment module 340 at a second flow rate to perform an extracorporeal treatment on the blood and returning the treated blood to a blood chamber 330.

In certain aspects, the method provides returning the blood from the T-junction 325 to the vascular access of the patient 316 at a third flow rate via conduit 318. The third flow rate is determined by the first blood pump 321. In certain aspects, the second flow rate is decoupled from both the first and third flow rates. In certain instances, the second flow is faster than the combination of both the first and the third flow rates.

In one embodiment, the present disclosure provides a blood treatment system, the system comprising:
a blood chamber for holding a batch of blood from a patient;
a first conduit for conveying blood from a vascular access of the patient to the blood chamber at a first flow rate, the first conduit having a first lumen;
a filter for performing extracorporeal treatment on blood passing therethrough by removing waste molecules, pathogens and/or fluid;
a recirculating blood processing loop connecting the blood chamber to the filter;
a first blood pump for conveying blood from the patient to the blood chamber;
a second blood pump for conveying blood through the recirculating blood processing loop at a second flow rate;
a third blood pump for conveying blood from the blood chamber to a second vascular access of the patient at a third flow rate via a second conduit, the second conduit having a second lumen; and
a controller configured to control the first blood pump for conveying blood from the patient to the blood chamber, the second blood pump for conveying blood through the recirculating blood processing loop and the third blood pump for conveying blood from the blood chamber to a second vascular access of the patient.

In another embodiment, the present disclosure provides a blood treatment system, the system comprising:
a blood chamber for holding a batch of blood from a patient;
a first conduit for conveying blood from a first vascular access of a patient to a first T-junction at a first flow rate, the conduit having a first lumen;
a first blood pump for conveying blood from the patient to a first T-junction;
a recirculating blood processing loop connecting the blood chamber and first T-junction to a filter and the filter to a second T-junction, wherein the second T-junction connects to the blood chamber;
a second blood pump for conveying blood through the recirculating blood processing loop at a second flow rate;
a third blood pump for conveying blood from the second T-junction to a second vascular access of the patient at a third flow rate via a second conduit, the second conduit having a second lumen; and
a controller configured to control the first blood pump for conveying blood from the patient to the first T-junction, the second blood pump for conveying blood through the recirculating blood processing loop and the third blood pump for conveying blood from the second T-junction to a second vascular access of the patient.

In yet another embodiment, the present disclosure provides a blood treatment system, the system comprising:
a blood chamber for holding a batch of blood from a patient;
a conduit for conveying blood from a vascular access of a patient to a T-junction at a first flow rate, the conduit having a lumen;
a first blood pump for conveying blood from the patient to the T-junction;
a recirculating blood processing loop connecting the blood chamber to a filter and the filter to the blood chamber;
a second blood pump for conveying blood through the recirculating blood processing loop at a second flow rate;
the first blood pump for conveying blood from the blood reservoir to the vascular access of the patient at a third flow rate via the conduit; and
a controller configured to control the first blood pump for conveying blood from the patient to the T-junction, the second blood pump for conveying blood through the recirculating blood processing loop and the first blood pump conveying blood from the blood chamber to the vascular access of the patient.

In certain aspects, the controller is configured to control operation of the first, second and/or third blood pumps in performing an extracorporeal treatment on the batch of blood from the patient.

In certain aspects, the controller is configured to control a blood pump to repeatedly recirculate blood from the blood chamber through the filter or treatment module.

In certain aspects, the systems herein comprise one or more valves operatively coupled to the controller. For example, the controller is configured to control one or more valves of the fluid circuit and the blood pump(s) to deliver at least one of supplemental fluid, anticoagulant, or anticoagulant agent from a respective source to a flowpath.

In some embodiments, the methods and systems disclosed can be used to process other body fluids. For example, accumulation of fluid in the abdominal cavity is called ascites. Ascites can be common with patients with cirrhosis, liver disease or congestive heart failure. When removing a body fluid such as ascites, a diuretic can also be administered. Commonly used diuretics include spironolactone (Aldactone) and/or furosemide (Lasix). When fluid accumulation cannot be treated optimally with diuretics and a salt restricted diet, patients may require a large amount of fluid be removed (paracentesis) for relief of symptoms. The disclosure includes methods and systems for treating ascites, by the withdrawal of ascites. Optionally, the withdrawn ascitic fluid can be concentrated and reinfused.

Embodiments of the disclosed subject matter provide extracorporeal blood treatment systems and methods that decouple the blood flow during treatment processing from the blood flow to/from the patient. As a result, higher blood flow rates during the treatment processing can be obtained for improved solute clearance, including increased clearance of middle molecules over conventional systems. Since the treatment processing is decoupled from the blood withdrawal and infusion, a lower blood flow rate can be used for withdrawal/infusion of blood, thereby enabling a smaller bore/diameter for the needle or lumen providing access to the patient's vascular system. Although the present disclosure uses “blood” as an exemplary body fluid, those of skill in the art will recognize that the systems and methods of the present disclosure are also useful for other body fluids such as blood, lymph, ascites, abdominal fluid, pleural fluid, organ fluid, spinal fluid, intestinal fluid or water. Similarly, although “vascular access” is an exemplary embodiment, a skilled artisan will recognize that abdominal access is needed for ascites, spinal canal access is needed for spinal fluid, and lymphatic access is need for lymph.

In certain aspects, the decoupling can be achieved by batch processing of blood. For example, a volume of blood is removed from the patient to a batch container. Blood in the batch container is subsequently processed by a treatment module, before being returned to the patient. Alternatively, a volume of blood is removed from the patient and is sent to the treatment module. In certain instances, the processed blood can be returned to a blood chamber and repeatedly processed by the treatment module (e.g., passing through the treatment module multiple times) to further improve solute clearance.

In some embodiments, the systems and methods utilize a small single lumen (e.g., smaller than either 7 French, such as 6, 5, 4, or 3 French or 17 gauge such as 16, 15, 14, 13, 12, 11, or 10 gauge) to draw, at a first flow rate, a “micro” batch of blood or body fluid (e.g., about 10-300 ml, or 2-7% such as about 2, 3, 4, 5, 6, or 7% of the patient's total blood volume) into a single reservoir. The volume of body fluid can be, for example, about 10 ml-100 ml, 10 ml to 200 ml, 10 ml to 300 ml, 10 ml to 400 ml, 10 ml to 500 ml, 10 ml to 600 ml, 10 ml to 700 ml, 10 ml to 800 ml, 10 ml to 900 ml or 10 ml-1000 ml. A batch of blood in the treatment circuit can be circulated at a higher second flow rate through a treatment module, such as hemofilter, hemodialyzer, or hemoperfusion device, thereby enabling efficient small and middle molecule clearance. After sufficient circulations, the blood is returned, at third flow rate (which may be the same as or different from the first flow rate) to the patient via the small single lumen. The cycle can then be repeated multiple times, for example, to process an entire blood volume of the patient.

In certain instances, the filter or filtration device can be for example, an extracorporeal hemoadsorption filter device to remove cytokines from circulating blood such as a biocompatible, sorbent bead technology e.g., CytoSorb™, CytoSorbents™, Inc. CytoSorb hemoadsorption beads are polystyrene-divinylbenzene porous particles (450 μm avg. particle diameter, 0.8-5 nm pore diameter, 850 m²/g surface area) with a biocompatible polyvinyl-pyrrolidone coating. See for example, U.S. Pat. No. 8,647,666 which claims a method of using a composition comprising polystyrene divinyl benzene copolymer and a polyvinyl pyrrolidone polymer.

In certain other instances, the filter or filtration device is Seraph® Microbind® Affinity Blood Filter, which is a filter that allows body fluids to pass over microbeads coated with molecular receptor sites that mimic the receptors on human cells which pathogens use to colonize when they invade the body. The adsorption media is a flexible platform that uses covalently-bonded, immobilized heparin or heparan sulfate for its unique binding capacity. See, for example, U.S. Pat. Nos. 8,758,286 or 9,173,989, disclosing at least one polysaccharide adsorbent, or immobilized heparin.

A blood treatment system, the system comprising:
a blood chamber for holding a batch of blood from a patient;
a first conduit for conveying blood from a vascular access of the patient to the blood chamber at a first flow rate, the first conduit having a first lumen;
a filter for performing extracorporeal treatment on blood passing therethrough by removing waste molecules, pathogens and/or fluid;
a recirculating blood processing loop connecting the blood chamber to the filter;
a first blood pump for conveying blood from the patient to the blood chamber;
a second blood pump for conveying blood through the recirculating blood processing loop at a second flow rate;
a third blood pump for conveying blood from the blood chamber to a second vascular access of the patient at a third flow rate via a second conduit, the second conduit having a second lumen; and
a controller configured to control the first blood pump for conveying blood from the patient to the blood chamber, the second blood pump for conveying blood through the recirculating blood processing loop and the third blood pump for conveying blood from the blood chamber to a second vascular access of the patient.

A blood treatment system, the system comprising:
a blood chamber for holding a batch of blood from a patient;
a first conduit for conveying blood from a first vascular access of a patient to a first T-junction at a first flow rate, the conduit having a first lumen;
a first blood pump for conveying blood from the patient to a first T-junction;
a recirculating blood processing loop connecting the blood chamber and first T-junction to a filter and the filter to a second T-junction, wherein the second T-junction connects to the blood chamber; a second blood pump for conveying blood through the recirculating blood processing loop at a second flow rate;
a third blood pump for conveying blood from the second T-junction to a second vascular access of the patient at a third flow rate via a second conduit, the second conduit having a second lumen; and
a controller configured to control the first blood pump for conveying blood from the patient to the first T-junction, the second blood pump for conveying blood through the recirculating blood processing loop and the third blood pump for conveying blood from the second T-junction to a second vascular access of the patient.

A blood treatment system, the system comprising:
a blood chamber for holding a batch of blood from a patient;
a conduit for conveying blood from a vascular access of a patient to a first T-junction at a first flow rate, the conduit having a lumen;
a first blood pump for conveying blood from the patient to a T-junction;
a recirculating blood processing loop connecting the blood chamber to a filter and the filter to the blood chamber;
a second blood pump for conveying blood through the recirculating blood processing loop at a second flow rate;
the first blood pump for conveying blood from the blood reservoir to the vascular access of the patient at a third flow rate via the conduit; and
a controller configured to control the first blood pump for conveying blood from the patient to the T-junction, the second blood pump for conveying blood through the recirculating blood processing loop and the first blood pump conveying blood from the blood chamber to the vascular access of the patient.

Features discussed herein can be performed on a single or distributed processor (single and/or multi-core), by components distributed across multiple computers or systems, or by components co-located in a single processor or system. For example, aspects of the disclosed subject matter can be implemented via a programmed general purpose computer, an integrated circuit device, (e.g., ASIC), a digital signal processor (DSP), an electronic device programmed with microcode (e.g., a microprocessor or microcontroller), a hard-wired electronic or logic circuit, a programmable logic circuit (e.g., programmable logic device (PLD), programmable logic array (PLA), field-programmable gate array (FPGA), programmable array logic (PAL)), software stored on a computer-readable medium or signal, an optical computing device, a networked system of electronic and/or optical devices, a special purpose computing device, a semiconductor chip, a software module or object stored on a computer-readable medium or signal.

When implemented in software, functions may be stored on or transmitted over as one or more instructions or code on a computer-readable medium. The steps of a method or algorithm disclosed herein may be embodied in a processor-executable software module, which may reside on a computer-readable medium. Instructions can be compiled from source code instructions provided in accordance with a programming language. The sequence of programmed instructions and data associated therewith can be stored in a computer-readable medium (e.g., a non-transitory computer readable medium), such as a computer memory or storage device, which can be any suitable memory apparatus, such as, but not limited to read- only memory (ROM), programmable read-only memory (PROM), electrically erasable programmable read-only memory (EEPROM), random-access memory (RAM), flash memory, disk drive, etc.

As used herein, computer-readable media includes both computer storage media and communication media, including any medium that facilitates the transfer of a computer program from one place to another. Thus, a storage media may be any available media that may be accessed by a computer. By way of example, and not limitation, such computer-readable media may comprise RAM, ROM, EEPROM, CD-ROM or other optical disk storage, magnetic disk storage or other magnetic storage devices, or any other medium that may be used to carry or store desired program code in the form of instructions or data structures and that may be accessed by a computer.

Also, any connection is properly termed a computer-readable medium. For example, if the software is transmitted from a website, server, or other remote source using a transmission medium (e.g., coaxial cable, fiber optic cable, twisted pair, digital subscriber line (DSL), or wireless technologies such as infrared, radio, and microwave), then the transmission medium is included in the definition of computer-readable medium. Moreover, the operations of a method or algorithm may reside as one of (or any combination of) or a set of codes and/or instructions on a machine-readable medium and/or computer-readable medium, which may be incorporated into a computer program product.

Any range described herein will be understood to include the endpoints and all values between the endpoints. Whenever “substantially,” “approximately,” “essentially,” “near,” or similar language is used in combination with a specific value, variations up to and including 10% of that value are intended, unless explicitly stated otherwise.

It is thus apparent that there is provided, in accordance with the present disclosure, extracorporeal blood treatment systems and methods employing batch processing. Many alternatives, modifications, and variations are enabled by the present disclosure. While specific examples have been shown and described in detail to illustrate the application of the principles of the present invention, it will be understood that the invention may be embodied otherwise without departing from such principles. For example, disclosed features may be combined, rearranged, omitted, etc. to produce additional embodiments, while certain disclosed features may sometimes be used to advantage without a corresponding use of other features. Accordingly, Applicant intends to embrace all such alternative, modifications, equivalents, and variations that are within the spirit and scope of the present invention.

What is claimed is:

1. A blood treatment method comprising:
(a) conveying a volume of blood via a first conduit from a first vascular access of a patient to a blood chamber at a first flow rate, the first conduit having a first lumen;
(b) conveying the blood from the blood chamber through an extracorporeal therapeutic device at a second flow rate to perform an extracorporeal treatment on the blood and returning the treated blood to the blood chamber; and
(c) returning the blood from the blood chamber to a second vascular access of the patient at a third flow rate via a second conduit, the second conduit having a second lumen;
wherein the second flow rate is decoupled from both the first and third flow rates.

2. The method of claim 1, wherein the second flow is faster than the combination of both the first and the third flow rates.

3. The method of claim 1, wherein the first conduit is a needle or cannula forming at least part of the vascular access.

4. The method of claim 3, wherein the catheter or needle of the first conduit has a size of either 2-11 French or 10-23 gauge.

5. The method of claim 1, wherein the extracorporeal treatment is at least one of hemodialysis, hemofiltration, hemodiafiltration, or hemoperfusion.

6. The method of claim 1, wherein the volume of blood is 2-7%, inclusive, of a total blood volume of the patient.

7. The method of claim 1, wherein the second flow rate is 300 ml/min-600 ml/min, inclusive.

8. The method of claim 1, wherein in step (b), middle molecules contained in the blood are removed via the extracorporeal therapeutic device.

9. The method of claim 1, wherein the first conduit comprises a single-lumen catheter or needle having a size smaller than either 7 French or 17 gauge; and
beta 2 microglobulin clearance is at least 100 ml/min.

10. The method of claim 1, further comprising monitoring a weight of the blood chamber or a volume level of the blood chamber and correlating the monitored weight to a stage of the dialysis process.

11. The method of claim 1, comprising, prior to (b), adding a first volume of supplemental fluid and/or adding a second volume of anticoagulant to the blood chamber.

12. A blood treatment method comprising:
(a) conveying a volume of blood via a first conduit from a first vascular access of a patient to a first T-junction at a first flow rate, the first conduit having a first lumen;
(b) conveying the blood from the first T-junction through an extracorporeal therapeutic device at a second flow rate to perform an extracorporeal treatment on the blood and returning the treated blood to a second T-junction; and
(c) returning the blood from the second T-junction to a second vascular access of the patient at a third flow rate via a second conduit, the second conduit having a second lumen, or alternatively,
(d) conveying the blood from the second T-junction to a blood chamber to reprocess the blood in the extracorporeal treatment, wherein the second flow rate is decoupled from both the first and third flow rates.

13. The method of claim 12, wherein the first and third flow rate is about 50-200 ml/min.

14. The method of claim 12, wherein the second flow is about 300-600 ml/min.

15. The method of claim 12, wherein the first conduit is a needle or cannula forming at least part of the vascular access.

16. The method of claim 12, wherein the catheter or needle of the first conduit has a size of either 2-11 French or 10-23 gauge.

17. The method of claim 12, wherein the extracorporeal treatment is at least one of hemodialysis, hemofiltration, hemodiafiltration, or hemoperfusion.

18. The method of claim 12, wherein the volume of blood is 2-7%, inclusive, of a total blood volume of the patient.

19. A blood treatment method comprising:
(a) conveying a volume of blood via a first conduit from a vascular access of a patient to a T-junction at a first flow rate, the conduit having a lumen;
(b) conveying the blood from the T-junction through an extracorporeal therapeutic device at a second flow rate to perform an extracorporeal treatment on the blood and returning the treated blood to a blood chamber;
(c) returning the blood from the blood chamber to the vascular access of the patient at a third flow rate, wherein the second flow rate is decoupled from both the first and third flow rates.

20. The method of claim 19, wherein the second flow is faster than the combination of both the first and the third flow rates.
